(19) United States
(12) Patent Application Publication
(10) Pub. No.: US 2017/0239198 A1
(43) Pub. Date: Aug. 24, 2017

(54) CAPSAICINOIDS AND USES THEREOF AS MEDICAMENTS

(71) Applicant: Mandishora Israel Muzari, Carrum Downs, Victoria (AU)

(72) Inventor: Mandishora Israel Muzari, Carrum Downs, Victoria (AU)

(21) Appl. No.: 15/505,152

(22) PCT Filed: Jul. 31, 2015
(86) PCT No.: PCT/AU2015/000454

§ 371 Date: Feb. 20, 2017

(30) Foreign Application Priority Data
Aug. 19, 2014 (AU) 2014903253

(51) Int. Cl. A61K 31/165; A61K 9/00; A61K 9/48; A61K 36/81

(52) U.S. Cl. CPC A61K 31/165; A61K 36/81; A61K 9/0053; A61K 9/48

(57) ABSTRACT

The present invention is directed towards a medicament comprising a capsaicinoid possessing antiviral activity and useful in the treatment of viral infections and in the treatment of HIV/AIDS. The medicament also possesses neuroplasticity-enhancing activity and is useful in the treatment of stress and other psychological conditions. Further, the medicament possesses anti-inflammatory activity and is useful in the treatment of arthritis. The medicament has also been shown to possess antibacterial activity.

### FIELD OF THE INVENTION

The present invention relates to capsaicinoids and uses thereof as medicaments.

### BACKGROUND

The world has, for years, reeled under the scourge of HIV/AIDS. These ailments have brought about much suffering to both the infected and the affected. They have torn down whole generations of reproductive aged people, and although the majority of statistics are confined to developing nations, the truth is that the whole world falls in the category of the affected.

In some cases, involvement in the effort to combat this infection and disease has been on a compassionate and humanitarian basis. Whichever way one looks at it,

HIV/AIDS has placed heavy financial demands on everyone in this world. For the infected in particular, the battle to extend life whilst fighting the infection is traumatising.

It is common knowledge that millions of dollars have gone into the research and production of drugs aimed, at least, to bringing relief to people with HIV/AIDS. The cost of the available drugs is prohibitive for many sufferers, to say the least. The drugs used for treatment of HIV/AIDS, such as anti-retrorvirals, are aimed at controlling the virus and supporting the immune system and as yet, no vaccine or cure has been found.

Further, stress is the body's method of reacting to challenges by way of sympathetic nervous system activation, which results in the fight or flight response. The body cannot sustain this state for long periods of time. Persistent distress that is not resolved through coping or adaptation can lead to chronic stress causing anxiety, depression and other psychological and physiological disorders such as memory problems, irritability, pain, nausea and changed sleeping habits.

During a stress response, the hypothalamus secretes various hormones, such as corticotrophin-releasing hormones, which stimulates the body's pituitary gland and initiates a heavily regulated stress response pathway. During stress, the hippocampus is particularly important, in that cognitive processes such as prior memories, can have a great influence on enhancing, suppressing, or even independently generating a stress response. The hippocampus though, is also an area in the brain that is susceptible to damage caused by chronic stress.

The prefrontal cortex can also become impaired during stress response as it performs its duties of regulating cognitive processes including planning, attention and problem solving through extensive connections with other brain regions. Further, during the body's fight-or-flight response, cortisol increases blood sugar through gluconeogenesis, suppresses the immune system and aids in fat and protein metabolism.

Stress dramatically reduces the ability of the blood brain barrier to block the transfer of chemicals, including hormones, from entering the brain from the bloodstream. Therefore, when corticosteroids are released into the bloodstream, they are immediately able to penetrate the brain and bind to first the mineralocorticoid receptor (MR) and then the glucocorticoid receptor (GR). As the GR begin to become activated, neurones in the amygdala, hippocampus, and prefrontal cortex become over stimulated. If the stress response continues and becomes chronic, the hyperactivity of the neurons begins to physically change the brain and have severe damaging effects on one's mental health.

As the neurons begin to become stimulated, calcium is released through channels in their cell membranes. Although initially this allows the cells chemical signals to continue to fire allowing nerve cells to remain stimulated, if this continues the cells will become overloaded with calcium leading to overfiring of neuron signals. The overfiring of the neurons is seen to the brain as a dangerous malfunction; therefore, the brain triggers the cells to shut down to avoid death due to over stimulation.

Decline in both neuroplasticity and long term potentiation (LTP) occurs in humans after experiencing levels of high continual stress. To maintain homeostasis, the brain is continuously forming new neural connections, reorganising its neural pathways, and working to fix damages caused by injury and disease. This keeps the brain vital and able to perform cognitive complex thinking. When the brain receives a distress signal, it immediately begins to go into overdrive. Neural pathways begin to fire and rewire at hyperspeed to help the brain understand how to handle the task at hand. Often, the brain becomes so intently focused on this one task that it is unable to comprehend, learn, or cognitively understand any other sensory information that is being thrown at it during this time. This over stimulation in specific areas and extreme lack of use in others causes several physiological changes in the brain, which overall reduce or even destroy the neuroplasticity of the brain.

Generally, selective serotonin reuptake inhibitors (SSRIs) have been used in the treatment of stress and anxiety. These are commonly associated with a number of adverse effects which are clearly undesirable and often similar to the conditions caused by the stress itself, such as nausea, agitation and insomnia.

Further again, arthritis, in its numerous forms, affects great numbers of people. Arthritis involves inflammation of one or more joints and is caused by trauma or infection to the joint, or age. Pain, swelling and stiffness are common symptoms of arthritis; less common but still possible symptoms include fatigue, weight loss and inability to sleep.

Current arthritis treatment methods include physical therapy, lifestyle changes, orthopaedic bracing and medications, including NSAIDs, corticosteroids and monoclonal antibodies. Physical treatments do not often provide good results for arthritis sufferers, lifestyle changes are difficult to maintain and medications involve many adverse effects.

The present invention attempts to overcome at least in part the aforementioned disadvantages of previous medicaments and treatments.

### SUMMARY OF INVENTION

In accordance with one aspect of the present invention, there is provided a medicament comprising a capsaicinoid.

The medicament may possess antiviral activity and be useful in the treatment of viral infections and in the treatment of HIV/AIDS.

The medicament may possess neuroplasticity-enhancing activity and be useful in the treatment of stress and other psychological conditions.

The medicament may possess anti-inflammatory activity and be useful in the treatment of arthritis.

The capsaicinoid may be capsaicin. The source of the capsaicin may be Capsicum annuum Bird's Eye.

In accordance with another aspect of the present invention, the dose of the medicament may be 550mg.

The dosage regimen of the medicament may comprise the consumption of a 550mg capsule.

A single dose may comprise one capsule and the regimen may comprise one dose per day. The dose may be consumed during a meal. The length of one dosage regimen may be 90 days.

The medicament may also possess wound healing, anti-fungal, antibacterial, pain relief, cellular regeneration, metabolic enhancement and skin condition activity as well as immune system activity by acting as an immuno-activator. The medicament may also be useful in the treatment of malaria, stomach ulcers, tuberculosis, bronchitis, constipation and eczema.

### DESCRIPTION OF PREFERRED EMBODIMENTS

The present invention relates to a medicament comprising a capsaicinoid. The medicament possesses antiviral activity and is useful in the treatment of viral infections and in the treatment of HIV/AIDS. The medicament of the present invention exerts a strong antiviral activity on human immunodeficiency virus (HIV). The activity of the medicament results in a dramatic increase in the CD4 count of HIV infected patients, either in conjunction with its antiviral activity, or as a result of their activity against the virus.

Moreover, the medicament of the present invention possesses neuroplasticity-enhancing activity and is useful in the treatment of stress and other psychological conditions. Due to the capability of the hippocampus in suppressing stress response and even independently generating responses, the present medicament has been found to be effective in influencing one's stress perception by enhancing the ability of the hippocampus to suppress stress response. Neuropeptide Y, a protein synthesised in the hypothalamus, acts as a chemical messenger in the brain and is implicated in anxiety and stress, specifically, stress resilience. The present medicament acts on the hypothalamus to enhance stimulation for the abundant production of Neuropeptide Y protein thereby enhancing stress resilience.

The present medicament effectively enhances neuroplasticity greatly. It has been advantageously found that when good concentration levels of capsaicinoids (those of the present invention) in the body are maintained, the result is that neuroplasticity and neurogenesis is greatly improved. The creation of new pathways for the purpose of re-establishing lost functionality is also speeded up and enhanced. The rejuvenating effects of capsaicin on the central nervous system is unprecedented.

Additionally, the release of the messenger cortisol by the adrenal gland into the bloodstream is for the purpose of instructing the body to redistribute energy to areas where it is needed most as the body deals with a stressor or stressors. The medicament of the present invention works to improve the efficiency of the body's metabolism, mainly because of its anti-inflammatory effect which greatly reduces stress (which may be caused by chronic inflammation). There are threshold levels of capsaicinoids that need to be in the bloodstream to achieve this. The present medicament achieves or exceeds those levels.

The medicament also possesses anti-inflammatory activity and is useful in the treatment of arthritis.

The capsaicinoid is obtained from their natural source, being the fruit of the Capsicum genus of plants, for example chillies. In accordance with a preferred embodiment of the present invention, bird's eye chillies (100,000 SHU) are used. The relative low cost of bird's eye chillies results in the added advantage of a relatively inexpensive method of treatment.

The preferred capsaicinoid of the medicament of the present invention is capsaicin, the chemical structure of which is shown below.

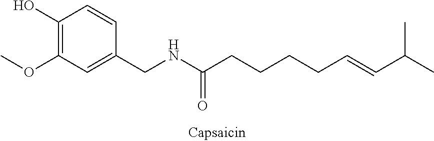 [SMILES: COc1cc(CNC(=O)CCCC/C=C/C(C)C)ccc1O]

The chillies are ground into a very fine powder and are then encapsulated for ingestion, in order to avoid contact with the mouth during ingestion. Preferably, each capsule is as small as is conveniently possible for manufacture. The small size of the capsule results in achieving the required amount of capsaicin to be administered, by varying the number of capsules ingested at each dosage point. It has been found that wider distribution in the stomach upon ingestion is achieved with a higher number of a smaller sized capsule. In turn, it has been found that this ensures a wider distribution of the active substance.

Following ingestion of the capsules, levels of capsaicin and/or other capsaicinoids in the bloodstream are found to be raised and those raised levels are sustained. The sustained levels of blood capsaicinoids are preferably those that are optimum for the antiviral or other therapeutic treatment according to the present invention.

The present medicament has been found to result in unprecedented relief and even recovery to patients infected with HIV. Patient trials have demonstrated that most participants who underwent this therapy tested negative for HIV after taking the medicament for a period of between three months and twelve months. Cases where patients' have gained an increase in CD4 count in excess of 400 cells/mm³, in just one month, have been recorded.

It is well known that capsaicin binds to the TRPV1 receptor, resulting in the signalling of heat and pain to the brain. This signalling is a result of merely the activation of TRPV1 and not any physical damage to cells. However, the sensation and discomfort that results, requires management for patients receiving capsaicinoid therapy.

The medicament of the present invention is preferably never consumed on an empty stomach. It is preferred that the medicament never be consumed without a meal, including not even shortly following a meal. More preferably, the medicament of the present invention is consumed at substantially the same time as a full meal. Most preferably, the medicament is consumed at approximately one quarter of the way through consumption of the full meal. In a preferred embodiment of the present invention, the full meal is an evening meal as drowsiness may occur, although the dosage may be consumed at any time of the day.

In use, each capsule preferably comprises 550 mg of finely ground bird's eye chillies. For the treatment of patients, one dose comprises one capsule. Each dose is taken with a meal once every day for 90 days. Preferably, the outer of the capsule disintegrates or dissolves at least 90 minutes following ingestion, allowing the contents of the capsule to be released in the lower parts of the stomach for easier management of the chilli heat.

In a preferred embodiment of the present invention, a minimum of 500 ml of water (preferably lukewarm, or according to a patient's preference), is consumed early each morning. This assists in both keeping hydrated and avoiding overly dry mucous membranes of the digestive tract. Greater volumes, above 500 ml, provide better effects.

After the 90 day regimen, the patient undergoes testing for the presence of their condition in a manner which would be understood by the skilled person. If a positive result is obtained, the same dosage regimen is continued. The patient undergoes similar testing every 30 days thereafter until a negative result is obtained.

It is preferred that, as large amounts of chillies are not harmful, patients continue taking the present medicament, even after testing negative for their condition. The continuing dosage may comprise a reduced quantity and frequency of capsules.

Other embodiment medicaments are also contemplated in accordance with the present invention. For instance, the aforementioned encapsulating of the plant material may, instead of forming a capsule, be provided as a coated table or, in fact, any method suited to the relevant function of providing a vehicle for delivery of the active ingredient while preventing discomfort to the patient. Likewise, instead of capsaicin, any suitable capsaicinoid, such as for example, dihydrocapsaicin or Nonivamide, to provide a favourable result, may be used. Both the size and volume of the capsule and the amount, frequency and length of the dosage regimen may be varied according to the optimal needs for the patient and the treatment. Further, while the preferred embodiment of the present medicament comprises bird's eye chillies, any appropriate source of capsaicinoid, which provides the desired treatment effect, may be used. It has been found that the higher the SHU rating of the capsaicinoid source (for example, a chilli with a higher SHU rating that bird's eye chillies), the greater effect achieved.

Modifications and variations as would be apparent to a skilled addressee are deemed to be within the scope of the present invention.

What is claimed is:

1-12. (canceled)

13. A medicament comprising a capsaicinoid possessing antiviral activity, when used in the treatment of viral infections and in the treatment of HIV/AIDS, characterised in that the medicament is consumed orally.

14. A medicament according to claim 13, characterised in that the medicament possesses neuroplasticity-enhancing activity, and is useful in the treatment of stress and other psychological conditions.

15. A medicament according to claim 13, characterised in that the medicament possesses anti-inflammatory activity, and is useful in the treatment of arthritis.

16. A medicament according to claim 13, characterised in that the capsaicinoid is capsaicin.

17. A medicament according to claim 16, characterised in that a source of the capsaicin is Capsicum annuum Bird's Eye.

18. A medicament according to claim 13, characterised in that the medicament possesses wound healing, anti-fungal, antibacterial, pain relief, cellular regeneration, metabolic enhancement and skin condition activity as well as immune system activity by acting as an immuno-activator.

19. A medicament according to claim 13, characterised in that the medicament is useful in the treatment of malaria, stomach ulcers, tuberculosis, bronchitis, constipation and eczema.

20. A treatment comprising the use of the medicament of claim 13, characterised in that a dose of the medicament is 550 mg.

21. A treatment according to claim 20, characterised in that the dose of the medicament comprises consumption of one capsule.

22. A treatment according to claim 20, characterised in that a dosage regimen comprises one dose per day.

23. A treatment according to claim 20, characterised in that the dose is consumed during a meal.

24. A treatment according to claim 20, characterised in that the dosage regimen may be 90 days in length.
